(19) United States
(12) Patent Application Publication
(10) Pub. No.: US 2010/0228144 A1
(43) Pub. Date: Sep. 9, 2010

(54) DEVICE FOR OCULAR STIMULATION AND DETECTIOIN OF BODY REACTIONS

(76) Inventor: Eric Labat, Marcq en Baroeuil (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/161,453

(22) PCT Filed: Jan. 19, 2007
(86) PCT No.: PCT/EP07/50559

§ 371 Date: Jul. 18, 2008

(30) Foreign Application Priority Data
Jan. 20, 2006 (FR) 0600514

(51) Int. Cl. A61B 5/00

(52) U.S. Cl. 600/558

(57) ABSTRACT

The invention relates to ocular stimulation and posturography equipment, characterized in that it comprises, in combination: a support capable of being removably fastened to the subject's head and including at least one ocular sight device to be placed facing one eye of the subject, each sight device comprising a display screen and a hollow body, in which the screen is placed, and being designed to be placed facing a single eye of the subject and to minimize the visual reference marks for the patient other than those displayed on the screen; means for detecting significant body reactions in the patient, which are capable of delivering measurement signals representative of significant body reactions; means for the acquisition and recording of the measurement signals delivered by the detection means; means for synchronizing the transmitted image signals and the received measurement signals so as to be able to correlate these two types of signal.

The present invention relates in a general manner to systems that are intended, during the ocular stimulation of a subject, to measure and record the movements of the body and the head, and any unbalance movements in particular, induced by this stimulation. A large and varied number of techniques currently exist to determine the posture of a human body.

One could mention, for example:
apparatus known as “statokinesigraphs” which, in an orthostatic position of the body, record random postural oscillations by means of a platform to which the individual is attached, and which is equipped with stress gauges.
dynamic posturography apparatus, which consist of a mechanical control system on a platform to which the individual is attached. The measurements are performed by means of electromyographs.
dynamic posturography apparatus that work with independent or correlated mechanical control loops on a platform with which a cabin is associated. In this case, the measurement is performed by a force platform.

The major drawbacks of these known apparatus are mainly their large size and their high cost. Besides, their applications are limited.

In particular, the application of the first apparatus mentioned in the examples is limited to static posturography.

One is also familiar with lighter systems for the execution of static or dynamic posturography, and in particular
apparatus in which luminous markers are applied to the cranium and the shoulders of a subject. The movement of these markers is then recorded by means of a photographic apparatus, the lens of which is aimed at a mirror attached to the ceiling of the room, and which records the trajectories described by the markers.

The application of such an apparatus is tiresome however, in particular due to the necessity to attach several luminous markers to the subject and to supply them with power, and to attach a mirror above the subject.
apparatus known as an “optoelectronic analyser”, in which a plurality of infrared reflectors are attached to the subject, and then exposed to infrared radiation. Several cameras of the charge-coupled device (CCD) type observe the subject and record the infrared radiation returned by the reflectors. This apparatus is extremely costly however, and its quality is a function of the number of cameras.
mask that is used to measure the movement of one eye of a patient in response to an optical stimulus. The optical stimulus is applied by means of image projectors located on the sides of the mask, and which project suitable images onto a mirror placed before the eyes of the patient (for further information on this, please see JP 05-049627). The image projection system is very bulky however, in addition to being complex to implement and requiring preliminary setting up of the projectors.
mask that measures the head movement of a patient in response to an optical stimulus. Measurement of the head movement is achieved by means of orthogonal accelerometers attached to the mask. The optical stimulus is delivered via a screen mounted in a hollow unit of rectangular section lying mainly on the optical axis of the eye of the patient (for further information, please see FR 2 760 348).
neurosensitive helmet that includes means to stimulate an eye, means to record the movement of the other eye using an infrared camera, and means to record the head movement by means of orthogonal accelerometers attached to the helmet (for further information, please see U.S. Pat. No. 4,817,633). In this case, ocular stimulation consists of an optokinetic module mounted on the helmet, in which the patient is able to see selected objects or graphics, and arranged to be opaque to all external light.

The present invention aims to propose a combined ocular stimulation and posturography device that is compact, simple to use, economic to manufacture, easy to position on a subject and to operate, ambulatory and that improves the quality of the measurements, the quality of the medical diagnosis and an appropriate re-education quality, in relation to the prior techniques.

Thus, the present invention proposes an ocular stimulation and posturography device, characterised in that it includes the following, in combination:
a support designed to be attached in a removable manner on the head of a subject and including at least one ocular sighting device to be placed before an eye of the subject, each sighting device including a display screen and a hollow body in which the screen is mounted, and which is arranged to be placed before a single eye of the subject and to minimise the visual references for the patient other than those displayed on the screen;
image signal control means, connected to the screen and designed to adjust at least the brightness, the nature, the direction of motion and the speed of appearance of patterns displayed on the screen with a view to stimulating the eye and thus provoking, in the patient, one or more body reactions that may be indicators of a medical symptom;
means to detect significant body reactions in the patient, adapted to supply measurement signals that represent significant body reactions;
means for acquisition and recording of the measurement signals supplied by the detection means;
means to synchronise the transmitted image signals and the received measurement signals so as to be able to correlate these two types of signal.

In addition, the absence of visual references at the stimulator, an appropriate choice of stimulating patterns to be presented to the subject, and synchronisation of the stimulation and detection signals, will improve the quality of the measurements, and in addition will ensure that the body reactions detected are indeed associated with medical symptoms, such as identification of visual dependency and its attenuation in the posture.

Preferred but non-limiting aspects of the equipment according to the invention are as follows:
the detection means include means to detect the eye movements, mounted on the said support of the device, before the eye which is not covered by the sighting device. This can be an infrared or pseudo-infrared camera for example. The means for detecting the eye movements can also be mounted on the said support in a manner that is removable and interchangeable with the said sighting device.
the detection means include means to detect the movements of at least one part of the body other than the eyes, these means being attached to the subject. A part of the body can be the head, these detection means then being attached to the said support, and can function with a set of acceleration sensors. In the latter case, the said acquisition means can include means for processing by integration, including double integrators respectively receiving the signals from the acceleration sensors.
the detection means include a force platform on which the patient stands upright.
the support of the ocular stimulation device includes a mask which, together with the face of the subject, creates a chamber that is essentially closed off from the light.
the hollow body of the said sighting device is designed so that its inside surface has no edge that is visible to the eye of the patient when the screen is displaying images.
the inside surface of the hollow body is black in colour and is in a matt material.
the sighting device also includes an electrical circuit connected to the screen and placed in the hollow body so as to be invisible to the eye of the patient.
the sighting device includes a light-tight seal located at the junction with the rest of the said support, as well as a hygienic seal that is disposable and replaceable after each use.
the sighting device is mounted in a removable manner on the said support.
the hollow body lies in a privileged direction, and the sighting device also includes adjustment means adapted to change the distance between the screen and the eye.
the support also includes at least one light source adapted to emit a light beam in a privileged direction chosen to be generally parallel to the ground when the subject wearing the support is standing upright, and used in particular for nuchal reeducation.
the equipment also includes a secondary housing attached to the subject and holding at least one part of the acquisition means, and a main housing holding control and recording means as well as means for processing and displaying the signals supplied by the acquisition means. The connection between the secondary housing and the main housing can be of the wireless type. The connection between the secondary housing and the detection means can be achieved using flexible wires. The main housing can include a case with compartments for the ocular stimulation device and its support and for the secondary housing.

Other aspects, aims and advantages of the present invention will appear more clearly on reading the detailed description that follows of a preferred embodiment of the latter, which is given by way of an example and with reference to the appended drawings in which:

FIG. 1 is a perspective view of a visual stimulation mask equipped with a posturography device according to the invention, worn by a subject,

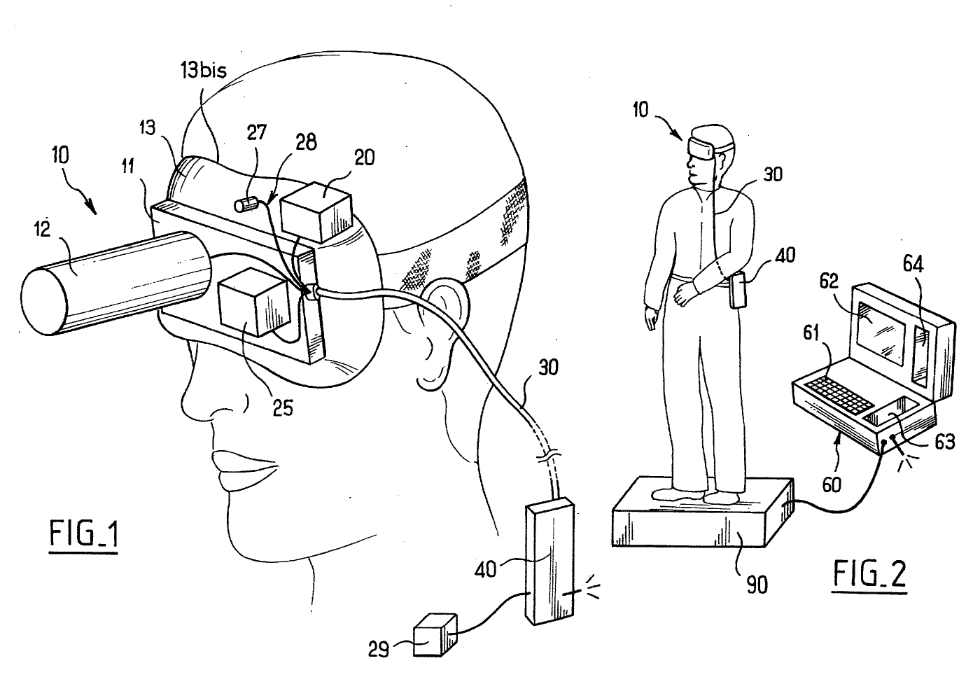

FIG. 2 is an overall view of the subject and of the posturography apparatus according to the invention,

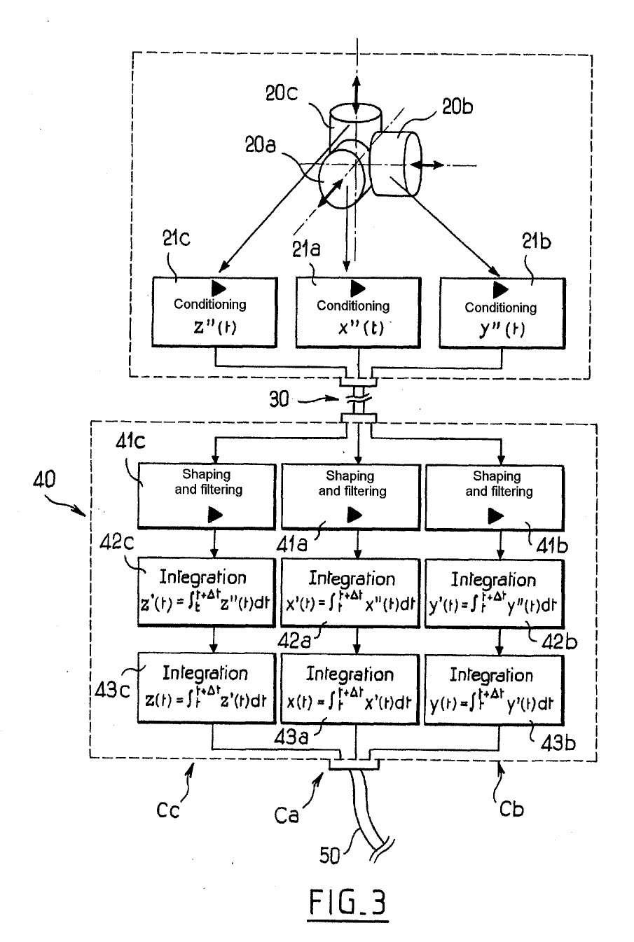

FIG. 3 is a block diagram of a part of the apparatus according to the invention, and

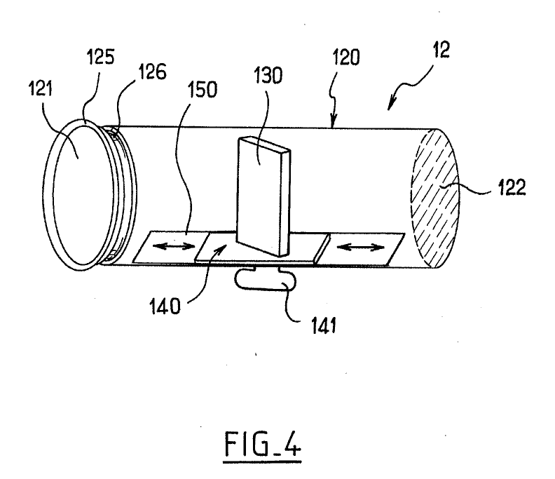

FIG. 4 is a perspective view of a sighting device according to the invention.

FIGS. 1 and 2 schematically illustrate an ocular stimulation device 10 that, in a manner that is conventional in its own right, includes a rigid framework 11 supporting one or two ocular stimulators that are capable of projecting stimulation images to infinity before the eye of the subject S. In this case, only a single stimulator has been shown, represented schematically as reference 12.

A flexible skirt 13 with its hygienic seal 13bis is placed between the framework 11 and the face of the subject to constitute a seal from the light and from dust and other impurities, and a strap is used to attach the assembly on the head of the subject. The hygienic seal 13bis is disposable and replaceable after each use.

According to the invention, and referring to FIG. 4, the stimulator is a sighting device 12 including a display screen 130 and a hollow body 120 in which the screen 130 is mounted, with the sighting device 12 being arranged so as to minimise the visual references to the patient other than those displayed on the screen 130.

In particular, the inside surface of the hollow body 120 is advantageously selected to have no edges at least in the part intended to be located between the screen 130 and the eye, and therefore having a section limited by a closed curve (such as an elliptical section or more particularly a disk) lying along a specified direction. This specified direction is advantageously straight ahead and is chosen to be in extension of the eye of the patient equipped with the mask. The hollow body 120 can thus have a cylindrical inside surface.

The hollow body 12 has an opening 121 that is intended to be positioned facing the eye, and that is also closed off by walls 122.

The inside surface of the hollow body 120 is advantageously black in colour, and in a matt material, reflecting very little of the light coming from the screen 130.

The hollow body 120 also includes a seal 125 around the opening 121 intended to provide a hermetic seal against the light at the junction between the sighting device 12 and the framework 11. Alternatively, the seal 125 can equally well be provided on the framework 11.

A hygienic seal that is disposable and replaceable after each use can also be provided. This seal can be separate or combined with seal 125.

Optionally, the sighting device 12 is mounted on the framework 11 in a removable manner. To this end, means for clipping, of screwing or other means 126 can be provided for fitting these two elements together in a light-tight manner.

The sighting device 12 can also include additional electrical circuits (not shown) designed to at least partially implement the screen 130. These circuits are advantageously placed in the device 12 so as not to be visible to the patient when the latter is wearing the mask 10. They can be hidden behind the screen for example 130.

The sighting device 12 can possibly include means that can be used to adjust the distance between the screen 130 and the eye.

These means can be telescopic in nature, and employed to change the length of the hollow body 120 (not illustrated), or means of the hinge 150 or slide 140 type attached to the screen 130 (shown in FIG. 4), the slide 140 being operated manually here by means of an external cursor 141 moving in a slot created through a wall of the device 12, this slot being substantially parallel to the principal axis of the hollow body 120. In the latter case, the patient is able to change the position of the screen 130, so as to adapter it to his or her focal plane for example.

The screen 130 employed can be an LCD, VFD or plasma display device.

To the framework 11 of the visual stimulation device is also attached, before the other eye of the patient, preferably an infrared camera or pseudo-infrared camera 25 in order to measure the eye movements in a manner that is known in its own right. One is familiar, for example, with such “oculometers”, manufactured by the Framiral™, Digital NystagView™, or Synapsys™ companies.

The principle can be based on detecting the iris pattern. The operator calibrates the centre of the eye by means of the camera 25, and the eye movements will then be observed.

In particular, the camera 25 can have as its objective to determine the triggering of a reflex nystagmus in a patient, where nystagmus consists of involuntary oscillatory movements of low amplitude and rotation of the eyeball. The physiological nystagmus, also known as optokinetic nystagmus, is liable to appear when the patient concentrates on a succession of images moving past him. It is composed of a series of slow jerking eye movements, following the object shown in movement, and a rapid shaking that seems to reset the eyeball.

The nystagmus can be evidence of a vestibular pathology by excitation or transient paralysis of one or both canals of the inner ear.

Also onto the head of the patient, at the framework 11 or the skirt 13, is attached a means for detecting the movement of the head 20. One can also arrange to attach to the wrist and/or to the hip for example, a means 29 to detect these parts of the body.

It is thus possible to measure the oscillations of the head and/or the movements of the body triggered by the reflex nystagmus for example, and to establish any possible correlation with a nystagmus.

These detection means 20-29 can include a gyroscope or a set of three accelerometric sensors 20a, 20b, 20c preferably oriented in the three directions of a orthonormal coordinate system, preferably with a forward/back direction, a left/right direction, and an up/down direction, in relation to the subject

It is advantageous to employ light and economical capacitive-effect accelerometers.

These three sensors deliver three raw signals that represent instantaneous accelerations experienced by each of the sensors, these signals then being applied to three buffer circuits 21a, 21b, 21c provided in the framework 11 of the mask 10 (not shown in FIG. 1). The outputs of these three buffers are transmitted via a set of flexible conductors 30 to an intermediate housing 40, attached to the belt of the subject for example, and containing processing circuits. The essential purpose of the buffer circuits 21a-21c is to perform impedance matching with a view to minimising disturbances in the signals on the line 30.

These processing circuits contained in the housing 40, and illustrated in FIG. 3, include three channels C1 to C3, where each channel respectively includes a signal shaping stage, amplification stage and filtering stage 41a to 41c, a first integration stage 42a to 42c, and a second integration stage 43a to 43c.

The amplification performed in stages 41a-41c is that intended to compensate for the low level of the received signals, while the filtration is advantageously effected with first-order low-pass analogue filters, in order to eliminate any parasitics.

The integration stages 42a-42c and 43a-43c also created in analogue technology, are used to obtain signals that represent the distances covered in the three dimensions by the head of the subject as a function of time, from the signals supplied by the accelerometers and processed upstream.

Optionally, the visual stimulation and posturography device according to the invention also includes a light source 27 attached to the framework 11 or the skirt 13, and adapted to emit a light beam mainly in a direction parallel to the ground when the patient wearing the mask is standing and erect.

This beam, projected remotely onto a support, can serve as a reference for the patient, in order that he can establish a specified direction.

This light beam can also help with nuchal reeducation.

The different detection means 20, 25 and 29 and the light source 27 are powered electrically, and supply the detection signals (for the detection means) by the use of flexible wires 28 that are then grouped within a cable 30 that is connected to a secondary housing 40.

This housing 40 is connected via a set of conductors or by a wireless link to a computer environment, equipped with an analogue input and analogue/digital conversion interface, this environment being designed to store, organise and display the collected movement data, and to perform comparisons with previous measurements.

This environment includes a local processing resource (such as a computer) and/or distant processing resource (such as a server).

The use of wireless connection will allow the patient to move freely. Such a device therefore improves the ambulatory character of the invention, and prevents accidents that can occur when the connecting wires are suddenly inadvertently pulled.

In addition, the invention proposes the addition of a force platform 90 adapted to detect a change in the pressure centre of the body of the patient on the platform 90, which can be indicative of a particular symptom. It is possible, for example, to employ a platform 90 manufactured by the Satel™ company.

The force platform 90 is connected to the said computer for processing of the acquired data.

The patient can thus step onto the force platform 90, standing in a comfortable position, namely with feet slightly apart, separated by 8 to 10 cm (calcaneal axis), for example, with arms relaxed alongside the body.

The platform 90 then records the movements of the body during the visual stimulation. The measurement is the movement of the pressure centre. The results can be expressed in terms of movement length and area and FFT spectral analysis.

A third person can be located near to the subject in order to prevent falls in the event that the subject experiences sensations of vertigo or balance disturbances as a result of the visual stimulations.

It is thus possible to measure the movements of the body, detected by the force platform 90 and the falls induced by the visual stimulations, and to establish any correlation with a nystagmus.

Advantageously, the processing effected by the distant and/or local processing means are as follows:
an additional, digital filtration of the received signals, in order to eliminate any residual parasitics and to selectively stop certain undesirable frequencies;
a spectral analysis by rapid breakdown into Fourier series;
statistical calculations, based, for example, on the extremes of movement, the entry and recording of accompanying data such as the name of the subject, the date, the time, etc. . . .
a display of data on the screen, with the ability to save these to a mass storage and restoration device, management of areas of interest by cursors, zooming, etc.

If the processing means is in the form of a computer, this can then be incorporated into an instrument case 60, the keyboard and the screen of this computer being designated by the references 61 and 62. This instrument case, which also includes compartments 63, 64 to hold the mask 10, the portable housing 40 and the different cables, allows one to be in possession of an apparatus that is compact and easy to transport.

In addition, due to the simplicity and low cost of the sensors and of the circuits employed, the apparatus of the invention can prove to be extremely economic in comparison with previous apparatus.

The powering of the different detection means 20, 25, 90 and light source 27 can be effected by batteries that are incorporated either into the mask 10 or into the portable housing 40, or into the instrument case 60. It can also be effected by the computer incorporated into the instrument case, which itself is powered from the mains supply.

In addition, it is thus possible to arrange for flexible electrical links 30 and wireless links between the mask 10 and the instrument case 60 are also used to transfer control signals for the sighting device 12 from the instrument case to the mask, and in particular video signals of images to be projected onto the eye of the subject, or indeed signals relating to direction and scrolling speed in the case of projecting recorded images onto a mobile support in the sighting device 12.

In this case, the movement signals supplied by the housing 40 are recorded in the computer in association with information representing the control signals.

These image signal control means connected to the screen 130, mounted in the computer, are adapted to adjust at least the brightness, the nature and the speed of appearance, and the direction of motion of patterns displayed on the screen 130 with a view to stimulating the eye, in order to provoke, in the patient, one or more body reactions that can be representative of a medical symptom, in particular so as to identify people who fall or are at risk of falling. These body reactions are then detected by the detection means assembly 20, 25, 29, 90.

It is possible, in particular, to employ patterns composed of chequered patterns, points, lines, simple or complex shapes, a planetarium, video image (virtual reality), and so on.

Means to synchronise the transmitted image signals with the received measurement signals, implemented by the processing means, are also provided in order to correlate these two types of signals and to then associate the detected body reactions with the different visual stimuli perceived by the eye.

Since the optokinetic stimulation of the patient, created from the said visual stimuli most often has a tendency to attract the subject in the direction of the rotation, postural adaptation reactions can be triggered.

The optokinetic stimulation and posturography according to the invention can therefore be used to diagnose the visual dependency of the posture.

In fact, certain subjects demonstrate excessive sensitivity to optokinetic stimulation, with inappropriate or excessive postural reactions, qualifying these as being visually dependent on posture.

Sensations of vertigo, balance disturbances and nausea, leading to falls, vomiting and fainting, are thus liable to appear.

Optokinetic stimulation and posturography according to the invention can be proposed in order to identify subjects who fall or are at risk of falling, which occur in elderly people, people with Parkinson's disease, those with central vestibular syndromes and/or those who have been victims of a stroke.

The knowledge and the diagnosis of this visual dependency character in a given patient can then influence the reeducation programme employed.

Thus, optokinetic stimulation and posturography according to the invention can also be used as a reeducation tool.

In particular, they can form an integral part of so-called vestibular reeducation programmes, so that after lesion of the vestibular system, most frequently after an acute and unilateral attack such as that caused by vestibular neuritis or surgery on the vestibular system.

They can also be used for reeducation after balance disturbances of neurological origin, such as vertigo, after cranial trauma or a stroke in particular.

The ability of the device according to the invention to trigger and to detect pathological body reactions therefore allow many applications in the study of pathologies, in diagnosis, in prevention and in reeducation, due in particular to its ease of use and its ambulatory character.

Because of the ambulatory character of the device, the invention can be used in particular to perform medical analyses other than in a specialist unit, such as in a hospital, a doctor's surgery or any other medical centre. It is thus possible even to use the device in the home.

1. An ocular stimulation and posturography device, characterised in that it includes, in combination:
a support (10, 11) adapted to be attached, in a removable manner, onto the head of a subject, and that includes at least one ocular sighting device (12) to be placed before an eye of the subject, where each sighting device (12) includes a display screen (130) and a hollow body (120) in which the screen is mounted (130), and which is arranged to be placed before a single eye of the subject and to minimise the visual references for the patient other than those displayed on the screen (130);
image signal control means connected to the screen (130), and adapted to adjust at least the brightness, the nature, the direction of motion and the speed of appearance of patterns displayed on the screen (130) with a view to stimulating the eye and thus provoking, in the patient, one or more body reactions that may be indicators of a medical symptom;
means (25, 20, 29, 90) to detect significant body reactions in the patient, which are adapted to supply measurement signals that represent significant body reactions;
means (40, 60) for acquisition and recording of the measurement signals supplied by the detection means (25, 20, 29, 90);
means to synchronise the transmitted image signals and the received measurement signals so as to be able to correlate these two types of signals.

2. Equipment according to claim 1, characterised in that the detection means include means (25) to detect the eye movements, mounted on the said support (11) of the device before the eye not covered by the sighting device (12).

3. Equipment according to claim 1, characterised in that the means for the detection of eye movements (25) include an infrared or pseudo-infrared camera.

4. Equipment according to claim 2, characterised in that the means for detecting the eye movements (25) are mounted on the said support (11) in a manner that is removable and interchangeable with the sighting device (12).

5. Equipment according to claim 1, characterised in that the detection means include means to detect the movements of at least one part of the body other than the eyes (20, 29), these means being attached to the subject.

6. Equipment according to claim 5, characterised in that the means for detecting the movements of at least one part of the body other than the eyes include means for detecting the movements of the head (20), these means being attached to the support (10, 11).

7. Equipment according to claim 5, characterised in that each means for detection of at least one part of the body other than the eyes (20, 29) functions with a set of acceleration sensors (20a, 20b, 20c).

8. Equipment according to claim 7, characterised in that the acquisition means include means for processing by integration (42a, 42b, 42c, 43a, 43b, 43c) including double integrators receiving the signals of the acceleration sensors (20a, 20b, 20c) respectively.

9. Equipment according to claim 1, characterised in that the detection means include a force platform (90) on which the patient stands upright.

10. Equipment according to claim 1, characterised in that the support of the ocular stimulation device includes a mask (10) which, together with the face of the subject, creates a chamber that is essentially closed off from the light.

11. Equipment according to claim 1, characterised in that the hollow body (120) of the said sighting device (12) is designed so that its inside surface has no edge visible to the eye of the patient when the screen (130) is displaying images.

12. Equipment according to claim 1, characterised in that the inside surface of the hollow body (120) is black in colour and is in a matt material.

13. Equipment according to claim 1, characterised in that the sighting device (12) also includes an electrical circuit (10) connected to the screen and placed in the hollow body (120) so as to be invisible to the eye of the patient.

14. Equipment according to claim 1, characterised in that the sighting device (12) includes a seal (125) to the light, located at the junction with the rest of the said support (10, 11), as well as a hygienic seal that is disposable and replaceable after each use.

15. Equipment according to claim 1, characterised in that the sighting device (12) is mounted in a removable manner on the said support (10, 11).

16. Equipment according to claim 1, characterised in that the hollow body (12) lies in a privileged direction, and in that the sighting device (12) also includes adjustment means (140, 141) adapted to change the distance between the screen (130) and the eye.

17. Equipment according to claim 1, characterised in that the support (10, 11) also includes at least one light source (27) adapted to emit a light beam in a privileged direction chosen to be being generally parallel to the ground when the subject wearing the support is standing erect, and in particular allowing nuchal reeducation.

18. Equipment according to claim 1, characterised in that it also includes a secondary housing (40) attached to the subject and holding at least one part of the acquisition means, and a main housing (60) holding control and recording means as well as means for processing and displaying the signals supplied by the acquisition means.

19. Equipment according to claim 1, characterised in that the connection between the secondary housing (40) and the main housing (60) is wireless.

20. Equipment according to claim 1, characterised in that the connection between the secondary housing (40) and the detection means (25, 20, 29) is achieved using flexible wires.

21. Equipment according to claim 1, characterised in that the main housing includes an instrument case (60) with compartment (63, 64, 10) for the ocular stimulation device and its support (10) and for the secondary housing (40).
